(19) United States
(12) Patent Application Publication
(10) Pub. No.: US 2025/0179430 A1
(43) Pub. Date: Jun. 5, 2025

(54) CELL GROWTH SUPPLEMENTS USING DAIRY-BASED MATERIALS

(71) Applicant: Terasaki Institute for Biomedical Innovation, Los Angeles, CA (US)

(72) Inventors: Samad Ahadian, Los Angeles, CA (US); Wei Huang, Sherman Oaks, CA (US); Alireza Khademhosseini, Los Angeles, CA (US)

(73) Assignee: Terasaki Institute for Biomedical Innovation, Los Angeles, CA (US)

(21) Appl. No.: 18/876,773

(22) PCT Filed: Jun. 29, 2023
(86) PCT No.: PCT/US2023/026523

§ 371 Date: Dec. 19, 2024

Related U.S. Application Data
(60) Provisional application No. 63/358,374, filed on Jul. 5, 2022.
(60) Provisional application No. 63/358,346, filed on Jul. 5, 2022.

(51) Int. Cl. C12N 5/071; C12N 5/074; C12N 5/077; C12N 5/0793

(52) U.S. Cl. CPC C12N 5/0631; C12N 5/0619; C12N 5/0653; C12N 5/0654; C12N 5/0656; C12N 5/0658; C12N 5/066; C12N 5/069; C12N 5/0696; C12N 2501/105; C12N 2501/998

(57) ABSTRACT

The present disclosure relates to cell growth supplements using dairy-based materials for cell culture and other applications. Some embodiments are directed to sequentially culturing cells in a culture medium comprising factors to increase the relative size and/or density of the cells in the culture, e.g., whey protein. Other aspects of the disclosure relate to obtaining a milk product to produce the dairy-based material, e.g., milking a nursing mammal, milk-producing organoids, bacterial expression systems, etc. In some embodiments, the disclosure relates to techniques for isolating a whey protein from the milk product.

### RELATED APPLICATIONS

This application claims the benefit of U.S. Provisional Patent Application Ser. No. 63/358,374, filed Jul. 5, 2022, entitled “Cell Growth Supplements Using Dairy-Based Materials,” and U.S. Provisional Patent Application Ser. No. 63/358,346, filed Jul. 5, 2022, entitled “Dairy-Based Microstructures as Microcarriers, Scaffolds, Substrates, and Other Applications,” each of which is incorporated herein by reference in its entirety.

### FIELD

The present disclosure relates to cell growth supplements using dairy-based materials for use in cell culture and other applications.

### BACKGROUND

Cells grown using in vitro cell culture systems are useful tools for the production of various cultivated products, including monoclonal antibodies, hormones, vaccines, and animal foods, among many others. In vitro cell cultures traditionally require a defined cell culture growth medium that supplies the necessary nutrients, e.g., lipids, vitamins, and amino acids, required for continued growth and proliferation. In addition, most cell culture growth mediums comprise growth factors to stimulate cells proliferate. The most commonly employed source for growth factors is fetal bovine serum (FBS); however, the supply of FBS is limited by the number of pregnant cows slaughtered each year and is often subject to undesirable batch-to-batch variations. Further, because FBS is a blood product, it may comprise pathogens, e.g., bacteria and/or viruses, that are harmful to mammalian cell culture. Despite over a decade of research, FBS remains the mainstay growth supplement for cell culture applications.

### SUMMARY

The present disclosure generally relates to cell growth supplements using dairy-based materials for cell culture and other applications. For example, in some embodiments, an animal-free cell culture growth medium capable of stimulating cellular proliferation and growth is provided. The subject matter of the present disclosure involves, in some cases, interrelated products, alternative solutions to a particular problem, and/or a plurality of different uses of one or more systems and/or articles.

For example, one aspect is generally directed to a method comprising exposing a plurality of cells to cell culture media comprising whey protein, and growing the plurality of cells in the cell culture media until the cells increase in size by an average of least 450%, relative to the size of the cells prior to exposing the cells to the cell culture media.

Another aspect is generally drawn to a method comprising exposing a plurality of cells to a first cell culture media and growing the cells in the first culture media for at least 24 hours, exposing the plurality of cells to a second cell culture media comprising a whey protein, and growing the plurality of cells in the second cell culture media until the cells increase in size by an average of least 450%, relative to the size of the cells prior to the second cell culture media.

In another aspect, the method comprises obtaining a milk product from a milk-producing mammal, extracting a whey protein from the milk product, forming cell culture media comprising the whey protein, exposing a plurality of cells to the cell culture media, and growing the plurality of cells in the cell culture media until the cells increase in size by an average of least 450%, relative to the size of the cells prior to exposing the cells to the cell culture media.

In yet another aspect, the method comprises obtaining a milk product from a milk-producing organoid, extracting a whey protein from the milk product, forming cell culture media comprising the whey protein, and exposing a plurality of cells to the cell culture media. In some cases, the organoid comprises a plurality of cultivated mammary epithelial cells.

The method, in still another aspect, comprises obtaining a whey protein from a fermenting bacterial culture, forming a cell culture media comprising the whey protein, and exposing a plurality of cells to the cell culture media.

In another aspect, the present disclosure encompasses methods of making one or more of the embodiments described herein, for example, dairy-based materials. In still another aspect, the present disclosure encompasses methods of using one or more of the embodiments described herein, for example, dairy-based materials.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a plot of absolute cell number of bovine skeletal muscle following 2 days of culture in various cell culture growth mediums, according to some embodiments;

FIG. 2A-E shows representative photomicrographs of cells cultured in various cell culture growth mediums using a live-dead assay.

FIG. 2F shows a plot of the average cell size of the cells grown in the various cell culture growth mediums; according to some embodiments;

FIG. 3A-F shows representative flow cytometry data used to quantify the size and density of bovine skeletal muscles cultured in the various cell culture growth mediums; according to some embodiments.

### DETAILED DESCRIPTION

The present disclosure relates to cell growth supplements using dairy-based materials for cell culture and other applications. Some embodiments are directed to sequentially culturing cells in a culture medium comprising factors to increase the relative size and/or density of the cells in the culture, e.g., whey protein. Other aspects of the disclosure relate to obtaining a milk product to produce the dairy-based material, e.g., milking a nursing mammal, milk-producing organoids, bacterial expression systems, etc. In some embodiments, the disclosure relates to techniques for isolating a whey protein from the milk product.

Certain aspects of the disclosure generally relate to the use of dairy-based materials for cell culture and other applications. In some embodiments, the dairy-based materials comprise a milk protein, e.g., whey protein or casein protein, etc. As discussed herein, milk proteins, e.g., whey protein, may be isolated from a milk product using techniques known to those of ordinary skill in the art, for example, acid-induced precipitation, salt-induced precipitation, and/or enzymatic treatment, which separates the milk product into a solid fraction and a liquid fraction. In some embodiments, the liquid fraction may be further purified to produce a whey protein.

In some embodiments, milk proteins, e.g., whey protein, may be processed into a microstructure, e.g., microparticles, and used, for example, as a cell culture substrate. In some cases, the microcarriers may be crosslinked or non-crosslinked. A binding agent, e.g., transglutaminase, may be used to at least partially crosslink the whey protein microstructures. In certain aspects of the disclosure, cells may be cultured on dairy-based microstructures, e.g., whey protein microparticles, using dairy-based products, e.g., soluble whey proteins.

In some aspects, the disclosure relates to methods for increasing the size and/or density of a culture of cells, e.g., mammalian cells, grown in a cell culture system, e.g., a bioreactor. The method, in some embodiments, may comprise culturing the cells in a first cell culture media comprising one or more factors, e.g., fetal bovine serum, that aid in cell adhesion to a scaffold (e.g., a culture dish or whey microcarrier). In some cases, the method comprises exchanging the first cell culture media with a second cell culture media comprising one or more factors, e.g., soluble whey protein, that increase the cell size and/or boost cellular proliferation of the adherent cells.

Some aspects of the disclosure generally relate to methods for obtaining a dairy-based material, e.g., whey protein. For example, in some cases, a dairy-based material may be produced from a milk product obtained, for example, by milking a pregnant and/or nursing mammal, e.g., a cow, sheep, goat, etc. The dairy-based material may also be obtained, for example, using engineered cells, e.g., bacterial fermentation, and engineered tissues, e.g., milk-producing organoids, according to other embodiments.

Other embodiments of the present disclosure generally relates to methods of producing one or more cultivated products using cell growth supplements comprising dairy-based materials. For example, in some embodiments, the dairy-based cell culture media may be used to increase the muscle mass of a biomass of skeletal muscle, for example, to produce a cell-based meat product. Other cultivated products may include, for example, textiles such as horn and leather, or medical products, such as artificial skin.

In some embodiments, exposing a plurality of cells cultured in a first cell culture growth media, which may comprise additives that promote cell adhesion, to a second cell culture growth media, comprising a dairy-based material, e.g., whey protein, may cause the cells to increase in size, relative to the size of the cells prior to exposure to the dairy-based material, e.g., whey protein. In some cases, culturing the plurality of cells to the dairy-based material, e.g., whey protein, induces the cells to undergo hypertrophic growth (i.e., increased volume). The extent of growth may generally correlate with the concentration of the dairy-based material in the cell culture growth media.

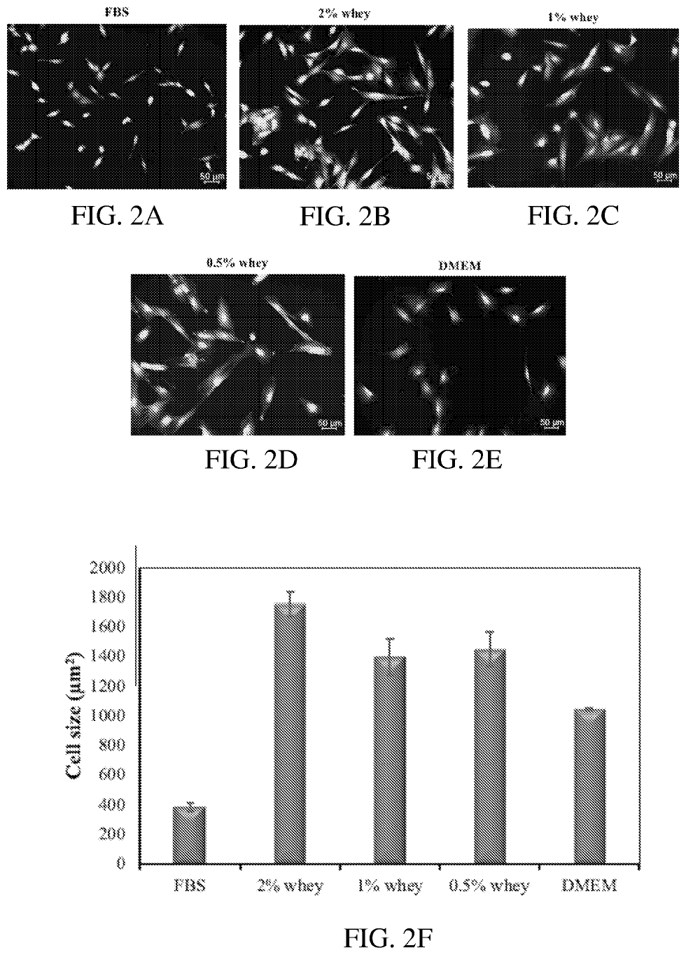

For example, cell culture growth media comprising 2% w/v whey protein may stimulate more hypertrophic growth than a cell culture growth media comprising 1% w/v whey protein, and so forth (see, e.g., FIG. 2). Any cell may undergo hypertrophic growth following exposure to the dairy-based material, e.g., muscle cells, fat cells, etc. In some embodiments, the hypertrophic cells may be larger because they produce more intracellular proteins and cytosol. For example, hypertrophic adipocytes may expand in size by depositing lipids within cytoplasmic vesicles, whereas hypertrophic skeletal muscles may grow by increasing the myofibril size.

Thus, in some embodiments, exposing a plurality of cells to a cell culture growth medium comprising a dairy-based material, e.g., whey protein, may cause the cells to increase in size. In some cases, the plurality of cells may first be cultured in a first cell culture growth medium, before being exposed to a second cell culture growth medium comprising a dairy-based material, e.g., whey protein. In some cases, the cells may increase in size by greater than or equal to 10%, by greater than or equal to 50%, by greater than or equal to 100%, by greater than or equal to 200%, by greater than or equal to 300%, by greater than or equal to 400%, by greater than or equal to 450%, by greater than or equal to 500%, by greater than or equal to 1000%, etc. In some embodiments, exposing a plurality of cells culture in a first cell culture growth medium to a second cell culture growth medium comprising a dairy-based material, e.g., whey protein, may cause the cells to increase in size by less than or equal to 1000%, by less than or equal to 500%, by less than or equal to 450%, by less than or equal to 400%, by less than or equal to 300%, by less than or equal to 200%, by less than or equal to 100%, by less than or equal to 50%, by less than or equal to 10%, etc. Combinations of any of these are possible in certain cases, e.g., the cells may increase in size between 300% and 400%, between 200% and 300%, between 50% and 200%, etc.

In addition, in some embodiments, exposing a plurality of cells cultured in a first cell culture growth media, comprising additives that promote cell adhesion, to a second cell culture growth media, comprising a dairy-based material, e.g., whey protein, may cause the cells to proliferate in a manner similar to traditional cell culture additives such as fetal bovine serum. The extent of proliferation may generally correlate with the concentration of the dairy-based material in the cell culture growth media.

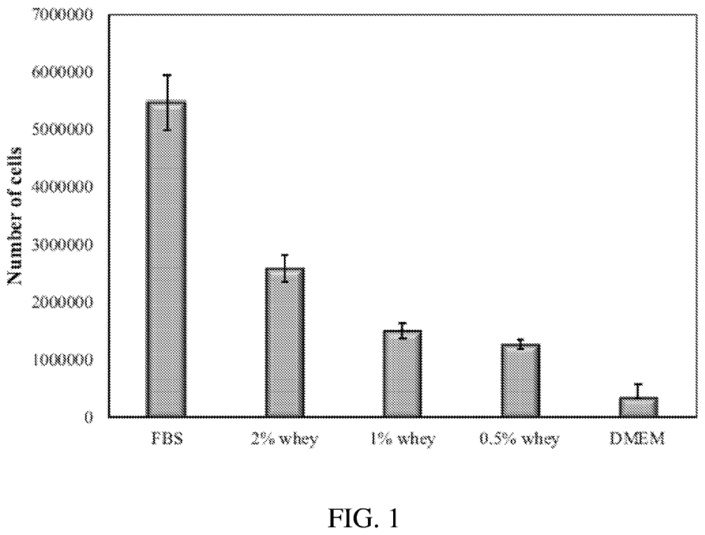

For example, cell culture growth media comprising 2% w/v whey protein stimulates more cell proliferation than a cell culture growth media comprising 1% w/v whey protein, and so forth (see, e.g., FIG. 1). In some embodiments, exposing a plurality of adherent cells (e.g., cells already attached to a substrate) to a second cell culture growth media comprising a dairy-based material, e.g., whey protein, between 0% w/v and 5% w/v may enhance cellular proliferation, for example, by greater than or equal to 1-fold, greater than or equal to 5-fold, greater than or equal to 10-fold, greater than or equal to 20-fold, etc., relative to cells cultured in a basal cell culture medium alone, e.g., DMEM. In some embodiments, exposing a plurality of adherent cells (e.g., cell already attached to a substrate) to a second cell culture growth media comprising a dairy-based material, e.g., whey protein (for example, between 0% w/v and 5% w/v) may enhance cellular proliferation, e.g., by less than or equal to 20-fold, less than to equal to 10-fold, less than or equal to 5-fold, less than or equal to 1-fold, etc., relative to cells cultured in a basal cell culture medium alone, e.g., DMEM.

In some embodiments, cells may be cultured in a first cell culture growth medium to allow the cells to adhere to a solid substrate. In some cases, the cells may be cultured in the first cell culture growth medium for greater than or equal to 6 hours, for greater than or equal to 12 hours, for greater than or equal to 24 hours, for greater than or equal to 48 hours, for greater than or equal to 72 hours. In some embodiments, the cells may be cultured in the first cell culture growth medium for less than or equal to 72 hours, less than or equal to 48 hours, less than or equal to 24 hours, less than or equal to 12 hours, less than or equal to 6 hours, etc. Combinations of these are also possible, e.g., the cells may be cultured for between 12 hours and 24 hours.

In some cases, a cell culture comprising a first cell culture growth medium may be serially diluted (i.e., multiple dilutions) with a second cell culture growth medium over a period of time, e.g., 1, 2, 3, or more days, for example, to slowly introduce a dairy-based material, e.g., soluble whey protein, into the cell culture growth medium (e.g., to allow the cells to acclimate to the new cell culture growth media). For example, in some embodiments, the cell culture may be grown in the first cell culture growth medium for greater than or equal to 12 hours, greater than or equal to 24 hours, greater than or equal to 48 hours, greater than or equal to 72 hours, or greater than or equal to 96 hours, e.g., before being serially diluted with the second cell culture growth medium. In some embodiments, the cell culture may be grown in the first cell culture growth medium for less than or equal to 96 hours, less than or equal to 72 hours, less than or equal to 48 hours, less than or equal to 24 hours, or less than or equal to 12 hours before being serially diluted with the second cell culture growth medium. Combinations are also possible, for example, in some embodiments, the cell culture may be diluted with a second cell culture growth medium after 12 hours and 24 hours, after 24 hours and 48 hours, after 12 hours and 48 hours, etc.

In some embodiments, a dairy-based material, e.g., a whey protein, may be present in a cell culture growth medium (e.g., a first and/or second cell culture growth medium) at any suitable concentration, for example, a concentration between 0% and 5% (w/v). In some instances, the concentration of the whey protein in the cell culture growth medium may be increased with the number of dilutions. For example, in some cases, a cell culture may be grown in a first cell culture growth medium comprising 0% whey protein for 24 hours, followed by a 50% dilution (v/v) with a second cell culture growth medium comprising 5% whey protein, thus increasing the final whey concentration to 2.5% in the cell culture after the first dilution. In some embodiments, the cell culture growth medium may comprise greater than or equal to 0% (w/v) dairy-based material, greater than or equal to 0.25% (w/v) dairy-based material, greater than or equal to 0.375% (w/v) dairy-based material, greater than or equal to 0.5% (w/v) dairy-based material, greater than or equal to 0.75% (w/v) dairy-based material, greater than or equal to 1.0% (w/v) dairy-based material, greater than or equal to 2.5% (v/v) dairy-based material, greater than or equal to 3.75% (w/v) dairy-based material, greater than or equal to 5% (w/v) dairy-based material, etc. In some embodiments, the cell culture growth medium comprises less than or equal to 5% (w/v) dairy-based material, less than or equal to 3.75% (w/v) dairy-based material, less than or equal to 2.5% (w/v) dairy-based material, less than or equal to 1.0% (w/v) dairy-based material, less than or equal to 0.75% (w/v) dairy-based material, less than or equal to 0.5% (w/v) dairy-based material, less than or equal to 0.375% (w/v) dairy-based material, less than or equal to 0.25% (w/v) dairy-based material, less than or equal to 0% (w/v) dairy-based material. Combinations of these are also possible in certain cases.

In some cases, an animal serum, e.g., a fetal bovine serum, may be present in a first and/or second cell culture growth medium, for example, at a concentration between 0% and 20% (w/v). In some instances, the concentration of the animal serum in the cell culture growth medium may be decreased with the number of dilutions performed. For example, in some cases, a cell culture may be grown in a first cell culture growth media comprising 10% (w/v) fetal bovine serum for 24 hours, followed by a 50% dilution (v/v) with a second cell culture growth media comprising 0% (w/v) fetal bovine serum, thus decreasing the final fetal bovine serum concentration to 5% (w/v) in the cell culture after the first dilution. In some embodiments, the first and/or second cell culture growth medium comprises greater than or equal to 0% (w/v) animal serum, greater than or equal to 5% w/v) animal serum, greater or equal to 7.5% (w/v) animal serum, greater than or equal to 10% (w/v) animal serum, greater than or equal to 20% (w/v) animal serum. In some embodiments, the first and/or second cell culture growth medium comprises less than or equal to 20% (w/v) animal serum, less than or equal to 10% (w/v) animal serum, less than or equal to 5% (w/v) animal serum, less than or equal to 0% (w/v) animal serum. Combinations of these are also possible in certain cases.

Some embodiments are generally directed to culturing cells in a cell culture system containing a solid substrate, e.g., microcarriers or tissue culture plate, and cell culture growth medium. In some cases, the cell culture growth medium is a first cell culture growth medium; in other cases, the cell culture growth medium is a second cell culture growth medium. In some embodiments, the first and/or second cell culture growth medium comprises one or more factors that promote cellular adhesion, proliferation, and/or hypertrophic growth of cells on the solid substrate. These may include any factor known to those of ordinary skill in the art, for example, various kids of growth factors. Exemplary factors include proteins (fibrinogen, fibronectin, laminin, vitronectin, collagen, etc.), proteoglycans (e.g., heparan sulfate, chondroitin sulfate, keratan sulfate, etc.), non-proteoglycan polysaccharides (e.g., hyaluronic acid), peptides (RGDS), polypeptides, nucleic acids (e.g., RNA or DNA), polynucleic acids, oligonucleotides, aptamers, growth factors (e.g., insulin growth factor type-1 and insulin growth factor type-2, etc.), or the like. Combinations of these and/or other factors are also possible; for example, in some cases, the cell culture growth medium may comprise laminin and insulin growth factor type-1; in another example, fetal bovine serum may be used in conjunction with insulin growth factor type-2, for example.

Other growth factors are also possible. Exemplary growth factors include, but are not limited to: adrenomedullin (AM), angiopoietin (Ang), autocrine motility factor, bone morphogenetic proteins (BMPs), ciliary neurotrophic factor (CNTF), leukemia inhibitory factor (LIF), interleukin-6 (IL-6), colony-stimulating factor (M-CSF, G-CSF, GM-CSF), epidermal growth factor (EGF), ephrins (A1-A5 and B1-B3), erythropoietin (EPO), fibroblast growth factor (FGF1-FGF23), fetal bovine somatotrophin (FBS), GDNF family of ligands (e.g., GDNF, neurturinn, persephin, artemin), growth differentiation factor-9 (GDF-9), hepatocyte growth factor (HGF), hepatoma-derived growth factor (HDGF), insulin, interleukins (IL-1-IL7), keratinocyte growth factor (KGF), migration-stimulation factor

(MSF), macrophage-stimulating protein (MSP), myostatin (GDF-8), neuregulins (NRG1-NRG4), neurotrophins (BDNF, NGF, NNT-3, NT-4), placental growth factor (PDGF), renalase (RNLS), T-cell growth factor (TCGF), thrombopoietin (TPO), transforming growth factors (TGF-alpha, TGF-beta), tumor necrosis factor-alpha (TNF-alpha), vascular endothelial growth factor (VEGF), wnt, etc. In some embodiments, the first cell culture media comprises a fetal bovine serum at a concentration between 0.01% and 10% (v/v).

Other sources of cell adhesion factors may also be possible. For example, in some embodiments, the cell adhesion factors may be harvested from the blood of non-human animals (for example, as described in Int. Pat. Apl. Nos. PCT/US22/19590, PCT/US22/19594, PCT/US22/19601, PCT/US22/19609, PCT/US22/19615, PCT/US22/19618, PCT/US22/19628, and PCT/US22/19631, each incorporated herein by reference in its entirety).

Certain embodiments are generally directed at culturing a plurality of cells, e.g., mammalian cells. In some cases, the plurality of cells may comprise myoblasts, fibroblasts, adipocytes, vascular cells, osteoblasts, tenocytes, epithelial cells, mammalian glands, neural cells, etc. Stem cells may also be cultured in some cases. Exemplary stem cells include, but are not limited to embryonic stem cells, induced pluripotent stem cells, and adult stem cells. Combinations are also possible (e.g., co-cultures of stem cells, co-cultures of stem cells with non-stem cells, co-cultures of two non-stem cells, etc.). The cells may arise from any animal species, including, but not limited to, human, monkey, cow or bovine, pig or swine, bison, elephant, whale, horse, deer, goat, crocodile, alligator, and/or camel.

Some embodiments are generally directed at culturing a plurality of cells, e.g., mammalian cells, to produce a cultivated product. In certain cases, the cultivated product may be a cell-based meat product, e.g., a ground beef product. For example, culturing the plurality of cells, e.g., muscle cells or fat cells, in a second cell culture growth media comprising a dairy-based product, e.g., whey protein, may cause the muscle cells to increase their volume between 10% and 1000% relative to cells not cultured in the same growth media. The muscle and fat cells may be mixed in various proportions to produce a meat product, e.g., a ground beef replica, without the known complications associated with large animal farming practices (e.g., foodborne illness, diet-related disease, antibiotic resistance, infectious disease, environmental degradation, and animal welfare concerns). Other cultivated products that may be produced using the methods of the current disclosure include horn, leather, wool, skin, organs, etc. Cultivated products, including cultivated food products, are further described in applications Int. Pat. Apl. Nos. PCT/US22/19590, PCT/US22/19594, PCT/US22/19601, PCT/US22/19609, PCT/US22/19615, PCT/US22/19618, PCT/US22/19628, and PCT/US22/19631, each incorporated herein by reference in its entirety.

Some embodiments of the current disclosure are directed towards obtaining a milk product, for example, to harvest the dairy-based material. In some cases, the milk product may be obtained from a milk-producing mammal, e.g., a human, cow, goat, sheep, etc. In some embodiments, the milk-producing mammal is a pregnant and/or nursing mammal. For example, dairy cows are continuously impregnated to ensure milk production for approximately 10 months of the year. In most cases, milk may be obtained from a milk-producing mammal by performing a plurality of milkings. Milk can be extracted from the mammary gland of the animal, e.g., cattle, water buffalo, humans, goats, sheep, camels, horses, donkeys, etc., e.g., by hand and/or machine. In some embodiments, the milk product is obtained from dairy cows. The average milk obtained from an average dairy cow may be greater than or equal to 5 L milk/day, greater than or equal to 7.5 L milk/day, greater than or equal to 10 L milk/day. In other embodiments, the average milk obtained from an average dairy cow is less than or equal to 10 L milk/day, less than or equal to 7.5 L milk/day, less than or equal to 5 L milk/day, etc. Combinations of these are also possible.

In some cases, milk and/or a milk product, e.g., whey protein, may be obtained from sources other than a pregnant mammal. For instance, in some embodiments, the milk product may be obtained from a culture of milk-producing organoids. Organoids are three-dimensional multicellular in vitro tissue constructs that mimic an in vivo organ function, e.g., milk production from breast tissue. Organoids may be produced using any appropriate cell type (e.g., embryonic and/or induced pluripotent stem cells). In some embodiments, the milk-producing organoids comprises functioning mammary epithelial cells that produce a milk product. In some embodiments, milk-producing organoids produce greater than or equal 0.001 ml/g, greater than or equal 0.002 ml/g, greater than or equal 0.003 ml/g, greater than or equal 0.005 ml/g, greater than or equal 0.01 ml/g, greater than or equal 0.02 ml/g, greater than or equal 0.03 ml/g, greater than or equal 0.05 ml/g, greater than or equal 0.1 ml/g, greater than or equal 0.2 ml/g, greater than or equal 0.3 ml/g, greater than or equal 0.5 ml/g, greater than or equal 1 ml/g, greater than or equal 2 ml/g, greater than or equal 3 ml/g, greater than or equal 5 ml/g, or greater than or equal 10 ml/g (per day). In other embodiments, the milk-producing organoids produce less than or equal to 10 ml/g, less than or equal to 5 ml/g, less than or equal to 3 ml/g, less than or equal to 2 ml/g, less than or equal to 1 ml/g, less than or equal to 0.5 ml/g, less than or equal to 0.3 ml/g, less than or equal to 0.2 ml/g, less than or equal to 0.1 ml/g, less than or equal to 0.05 ml/g, less than or equal to 0.03 ml/g, less than or equal to 0.02 ml/g, less than or equal to 0.01 ml/g, less than or equal to 0.005 ml/g, less than or equal to 0.003 ml/g, less than or equal to 0.002 ml/g, less than or equal to 0.001 ml/g, etc. Combinations of these are also possible.

In other cases, milk and/or a milk-product, e.g., a whey protein, may be obtained using an expression system, e.g., bacteria, yeast, insect, mammalian cells, etc., using recombinant DNA techniques. For example, in some embodiments, a gene encoding a whey protein of interest, e.g., an acidic whey protein, may be synthesized and incorporated into an expression plasmid (e.g., bacteria, yeast, insect, mammalian cells, etc.,). As an example, in some cases, the gene of interest may comprise a fusion tag, such as a histidine tag, e.g., for purification. In another non-limiting example, the gene of interest is operably linked to a promoter, e.g., lac promoter, that regulates expression of the gene of interest. The promoter may be a constitutively activated promoter or an inducible promoter (e.g., induced to the active state, for example, in response to an added drug such as doxycycline). In some embodiments, the expression plasmid is transfected into an expression system comprising one or more living cells e.g., bacteria cells, plant cells, yeast cells, insect cells, mammalian cells, etc., and the cells allowed to grow. As the cells grow, the gene of interest is transcribed into RNA and translated into the protein of interest, e.g., whey protein.

In some embodiments, a cell culture system, comprising an expression system comprising a gene of interest encoding a protein of interest, may be induced to stimulate production of the protein of interest, e.g., whey protein. Any cell culture system may be used, such as a tissue flask or a bioreactor (e.g., a fermenter). In some cases, the cell culture system may be used to produce greater than or equal to 0.5 L of culture, greater than or equal to 1 L of culture, greater than or equal to 2 L of culture, greater than or equal to 10 L of culture, greater than or equal to 100 L of culture, greater than or equal to 1000 L of culture, etc. In some embodiments, the cell culture system may be used to produce less than or equal to 1000 L of culture, less than or equal to 100 L of culture, less than or equal to 10 L of culture, less than or equal to 2 L of culture, less than or equal to 1 L of culture, less than or equal to 0.5 L of culture, etc. Combinations of these are also possible, e.g., the volume may be between 100 L and 1000 L, between 1 L and 2 L, between 10 L and 1000 L, etc. In some cases, the cells within the culture are harvested, for example, using centrifugation, lysed to release their intracellular contents, and the protein of interest isolated, for example, using techniques known to those of ordinary skill in the art (e.g., a nickel chromatography column configured to bind to a histidine fusion protein).

Some aspects of the disclosure are directed toward isolating a dairy-based material, e.g., a whey protein, from a whole milk product. The whey protein may be isolated from the whole milk product using any technique known to those of ordinary skill in the art. For example, in some embodiments, a milk product may be pasteurized, e.g., heated, to kill any pathogenic materials, e.g., bacteria, viruses, etc. In some cases, the pasteurized milk product may be treated with an enzyme, e.g., chymosin, that causes the milk product to curdle into a solid fraction (e.g., which may be used to produce different cheeses) and a liquid whey fraction. The liquid whey fraction may be filtered through one or more microfilters, according to certain embodiments, to remove fat and lactose from the whey component, for example, to yield a whey protein concentrate. In some cases, the liquid fraction may be further purified using ultrafiltration (e.g., size exclusion chromatography) to remove at least some of the fat and/or lactose, e.g., to yield a whey protein isolate.

The liquid whey protein concentration or isolate, in some cases, may be spray dried, e.g., using a commercial spray drier, to produce a whey protein powder. Spray drying ensures the whey protein does not get denatured during the drying process and retains its nutritional value. The spray dried whey protein may be dissolved in any basal media and used as a cell culture growth medium, for example, to increase the size of the cultured cells.

In certain embodiments, fabricating the dairy-based microstructures, e.g., microparticles, comprising whey protein uses a process known as salt-induced precipitation. Salt-induced precipitation (also known as salting out, salt fractionation, antisolvent crystallization, precipitation crystallization, or drowning out) is a technique that utilizes the reduced solubility of certain molecules in a solution of very high ionic strength. In some embodiments, the whey protein is hydrated in an aqueous solution, e.g., phosphate buffered saline to produce a hydrated whey protein solution. Following hydration, according to some embodiments, the whey protein solution may be heated to a temperature of at least 50° C., at least 60° C., at least 70° C., at least 80° C., at least 90° C., at least 100° C., etc., for at least 5 minutes, at least 10 minutes, at least 15 minutes, at least 20 minutes, at least 30 minutes, at least 45 minutes, at least 60 minutes, etc. Thereafter, the pH of the whey protein solution may be adjusted to a pH of at least 6.0, of at least 6.5, of at least 7.0, of at least 7.5, of at least 8.0, of at least 8.5, etc.

In some cases, a sodium chloride solution can be added to the whey protein solution, thus inducing the precipitation of microstructures. In some embodiments, the sodium chloride solution may have a concentration in the range of between 50 microM and 2.5 M. For example, in some embodiments, the sodium chloride concentration may be greater than or equal to 50 microM, greater than or equal to 100 microM, greater than or equal to 1 mM, greater than or equal to 1 M, greater than or equal to 2 M, greater than or equal to 2.5 M, etc. In some embodiments, the sodium chloride concentration less than or equal to 2.5 M, less than or equal to 2 M, less than or equal to 1 M, less than or equal to 1 mM, less than or equal to 100 microM, less than or equal to 50 microM, etc.

U.S. Provisional Patent Application Ser. No. 63/358,374, filed Jul. 5, 2022, entitled “Cell Growth Supplements Using Dairy-Based Materials,” and U.S. Provisional Patent Application Ser. No. 63/358,346, filed Jul. 5, 2022, entitled “Dairy-Based Microstructures as Microcarriers, Scaffolds, Substrates, and Other Applications,” each are incorporated herein by reference in its entirety.

The following examples are intended to illustrate certain embodiments of the present disclosure, but do not exemplify the full scope of the disclosure.

### EXAMPLE 1

In this example, the size and density of bovine skeletal muscle cells cultured in DMEM comprising either FBS or varying concentrations of whey protein were determined. A cheese whey was obtained as an end product of cheese manufacturing. The whey powder was suspended in DMEM cell culture growth media at different concentrations (e.g., 2%, 1% and 0.5%, w/v). Undissolved material was removed using centrifugation.

The cells were isolated and prepared using standard techniques. Freshly isolated cells were resuspended in DMEM basal media supplemented with 10% (v/v) and 1% (v/v) penicillin-streptomycin and seeded on to 25 cm² flasks at density of 2000 cells/cm². The flasks were placed in a humidified incubator in 5% CO₂ and 37° C.

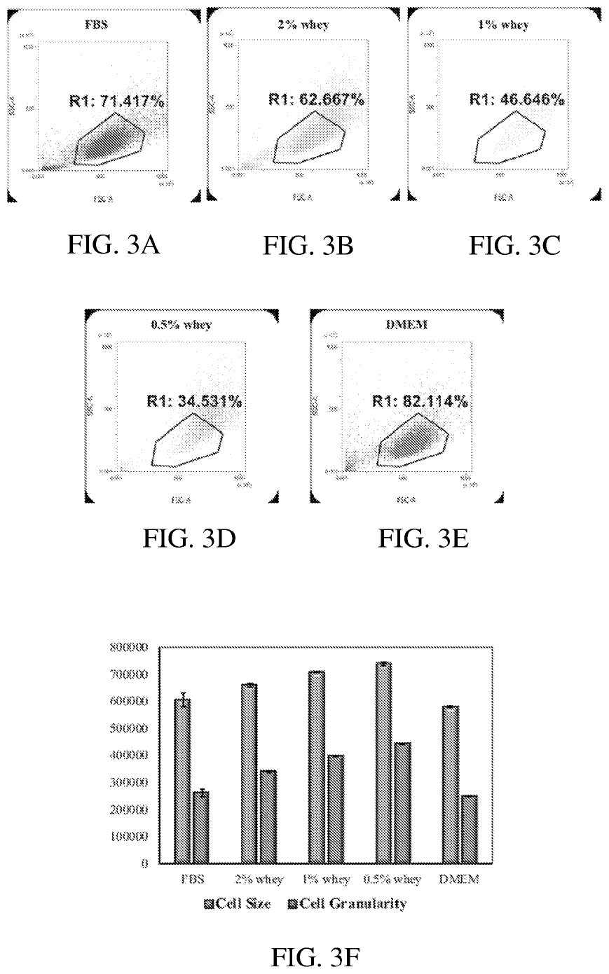

After 24 hours, the cell culture media was removed and the flasks were randomly assigned to receive media comprising 10% FBS/DMEM, whey protein/DMEM, or DMEM alone. Flasks assigned to the whey protein/DMEM group had their cell culture medium progressively replaced with either 2% whey protein/DMEM, 1% whey protein/DMEM, or 0.5% whey protein/DMEM over a 3-day period. For example, on day 0, the final composition of the cell culture media comprised 50% (v/v) of 10% FBS/DMEM and 50% (v/v) whey protein/DMEM; on day 1, the final composition of the cell culture composition comprised 25% (v/v) 10% FBS/DMEM and 75% (v/v) whey protein/DMEM; and on day 2, the final composition comprised 100% whey protein/DMEM. At various days, representative cells were taken and analyzed using a combination of fluorescent microscopy (FIGS. 1 and 2) and flow cytometry (FIG. 3).

The results showed that the cells were able to proliferate in whey protein/DMEM, with 2% whey yielding the greatest degree of proliferation. The results, also showed, unexpectedly, that culturing the cells with whey-based growth media significantly increased the overall size of the individual cells (FIGS. 2 and 3).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
exposing a plurality of cells to cell culture media comprising whey protein; and
growing the plurality of cells in the cell culture media until the cells increase in size by an average of least 450%, relative to the size of the cells prior to exposing the cells to the cell culture media.

2. The method of claim 1, wherein the cell culture media further comprises a growth factor.

3. The method of claim 2, wherein the growth factor comprises insulin growth factor-1.

4. The method of any one of claim 2 or 3, wherein the growth factor comprises insulin growth factor-2.

5. The method of any one of claims 1-4, wherein the plurality of cells comprises myoblasts.

6. The method of any one of claims 1-5, wherein the plurality of cells comprises fibroblasts.

7. The method of any one of claims 1-6, wherein the plurality of cells comprises adipocytes.

8. The method of any one of claims 1-7, wherein the plurality of cells comprises vascular cells.

9. The method of any one of claims 1-8, wherein the plurality of cells comprises osteoblasts.

10. The method of any one of claims 1-9, wherein the plurality of cells comprises tenocytes.

11. The method of any one of claims 1-10, wherein the plurality of cells comprises epithelial cells.

12. The method of any one of claims 1-11, wherein the plurality of cells comprises mammalian gland cells.

13. The method of any one of claims 1-12, wherein the plurality of cells comprises neural cells.

14. The method of any one of claims 1-13, wherein the plurality of cells comprises embryonic stem cells.

15. The method of any one of claims 1-14, wherein the plurality of cells comprises induced pluripotent stem cells.

16. The method of any one of claims 1-15, wherein the plurality of cells comprises adult stem cells.

17. The method of any one of claims 1-16, wherein the plurality of cells comprises human cells.

18. The method of any one of claims 1-17, wherein the plurality of cells comprises monkey cells.

19. The method of any one of claims 1-18, wherein the plurality of cells comprises cow cells.

20. The method of any one of claims 1-19, wherein the plurality of cells comprises sheep cells.

21. The method of any one of claims 1-20, wherein the plurality of cells comprises pig cells.

22. The method of any one of claims 1-21, wherein the plurality of cells comprises chicken cells.

23. The method of any one of claims 1-22, wherein the plurality of cells comprises goat cells. 24 The method of any one of claims 1-23, wherein the plurality of cells comprises bison cells.

25. The method of any one of claims 1-24, wherein the plurality of cells comprises elephant cells.

26. The method of any one of claims 1-25, wherein the plurality of cells comprises whale cells.

27. The method of any one of claims 1-26, wherein the plurality of cells comprises horse cells.

28. The method of any one of claims 1-27, wherein the plurality of cells comprises deer cells.

29. The method of any one of claims 1-28, wherein the plurality of cells comprises crocodile cells.

30. The method of any one of claims 1-29, wherein the plurality of cells comprises alligator cells.

31. The method of any one of claims 1-30, wherein the plurality of cells comprises camel cells.

32. The method of any one of claims 1-31, further comprising obtaining the whey protein from a milk product.

33. The method of claim 32, comprising obtaining the milk product from a mammal.

34. The method of claim 33, wherein the mammal is a pregnant mammal.

35. The method of claim 33, wherein the mammal is a nursing mammal.

36. The method of claim 32, comprising obtaining the milk product from a milk-producing organoid.

37. The method of claim 36, wherein the milk-producing organoid comprising a plurality of mammary epithelial cells.

38. The method of claim 32, comprising obtaining the milk product from a milk-producing bacterial fermentation culture.

39. The method of any one of claims 32-38, comprising adding a salt solution to the whey protein to precipitate the whey protein to form the milk product.

40. The method of claim 39, wherein the salt solution comprises sodium chloride is at a concentration of between 50 micromolar and 2.5 M.

41. The method of any one of claims 1-40, further comprising exposing a plurality of cells to a first cell culture media and growing the cells in the first culture media for at least 24 hours, prior to exposing the plurality of cells to the cell culture media comprising the whey protein.

42. The method of claim 41, wherein the first cell culture media comprises fetal bovine serum.

43. The method of claim 42, wherein the fetal bovine serum concentration is between 0.01% and 10% by mass.

44. The method of any one of claims 41-43, wherein the first cell culture media comprises a growth factor.

45. The method of claim 44, wherein the growth factor comprises insulin growth factor-1.

46. The method of any one of claim 44 or 45, wherein the growth factor comprises insulin growth factor-2.

47. A method, comprising:
exposing a plurality of cells to a first cell culture media and growing the cells in the first culture media for at least 24 hours;
exposing the plurality of cells to a second cell culture media comprising a whey protein; and
growing the plurality of cells in the second cell culture media until the cells increase in size by an average of least 450%, relative to the size of the cells prior to the second cell culture media.

48. The method of claim 47, wherein the first cell culture media comprises fetal bovine serum.

49. The method of claim 48, wherein the fetal bovine serum concentration is between 0.01% and 10% by mass.

50. The method of any one of claims 47-49, wherein the first cell culture media comprises a growth factor.

51. The method of claim 50, wherein the growth factor comprises insulin growth factor-1.

52. The method of any one of claim 50 or 51, wherein the growth factor comprises insulin growth factor-2.

53. The method of any one of claims 47-52, wherein the second cell culture media comprises between 0.5% and 2%, by mass, of the whey protein.

54. The method of claim 53, wherein the second cell culture media comprises the fetal bovine serum.

55. The method of claim 54, wherein the fetal bovine serum concentration is between 0.01% and 0.5% by mass.

56. The method of any one of claims 47-55, wherein the second cell culture media further comprises a growth factor.

57. The method of claim 56, wherein the growth factor comprises insulin growth factor-1.

58. The method of any one of claim 56 or 57, wherein the growth factor comprises insulin growth factor-2.

59. The method of any one of claims 56-58, wherein adding the second cell culture media to the plurality of cells increases the density of cells.

60. The method of any one of claims 47-59, wherein the plurality of cells comprises myoblasts.

61. The method of any one of claims 47-60, wherein the plurality of cells comprises fibroblasts.

62. The method of any one of claims 47-61, wherein the plurality of cells comprises adipocytes.

63. The method of any one of claims 47-62, wherein the plurality of cells comprises vascular cells.

64. The method of any one of claims 47-63, wherein the plurality of cells comprises osteoblasts.

65. The method of any one of claims 47-64, wherein the plurality of cells comprises tenocytes.

66. The method of any one of claims 47-65, wherein the plurality of cells comprises epithelial cells.

67. The method of any one of claims 47-66, wherein the plurality of cells comprises mammalian gland cells.

68. The method of any one of claims 47-67, wherein the plurality of cells comprises neural cells.

69. The method of any one of claims 47-68, wherein the plurality of cells comprises embryonic stem cells.

70. The method of any one of claims 47-69, wherein the plurality of cells comprises induced pluripotent stem cells.

71. The method of any one of claims 47-70, wherein the plurality of cells comprises adult stem cells.

72. The method of any one of claims 47-71, wherein the plurality of cells comprises human cells.

73. The method of any one of claims 47-72, wherein the plurality of cells comprises monkey cells.

74. The method of any one of claims 47-73, wherein the plurality of cells comprises cow cells.

75. The method of any one of claims 47-74, wherein the plurality of cells comprises sheep cells.

76. The method of any one of claims 47-75, wherein the plurality of cells comprises pig cells.

77. The method of any one of claims 47-76, wherein the plurality of cells comprises chicken cells.

78. The method of any one of claims 47-77, wherein the plurality of cells comprises goat cells.

79. The method of any one of claims 47-78, wherein the plurality of cells comprises bison cells.

80. The method of any one of claims 47-79, wherein the plurality of cells comprises elephant cells.

81. The method of any one of claims 47-80, wherein the plurality of cells comprises whale cells.

82. The method of any one of claims 47-81, wherein the plurality of cells comprises horse cells.

83. The method of any one of claims 47-82, wherein the plurality of cells comprises deer cells.

84. The method of any one of claims 47-83, wherein the plurality of cells comprises crocodile cells.

85. The method of any one of claims 47-84, wherein the plurality of cells comprises alligator cells.

86. The method of any one of claims 47-85, wherein the plurality of cells comprises camel cells.

87. The method of any one of claims 47-86, further comprising obtaining the whey protein from a milk product, and forming the second cell culture media comprising the whey protein.

88. The method of claim 87, comprising obtaining the milk product from a mammal.

89. The method of claim 88, wherein the mammal is a pregnant mammal.

90. The method of claim 88, wherein the mammal is a nursing mammal.

91. The method of claim 87, comprising obtaining the milk product from a milk-producing organoid.

92. The method of claim 91, wherein the milk-producing organoid comprising a plurality of cultivated mammary epithelial cells.

93. The method of claim 87, comprising obtaining the milk product from a milk-producing bacterial fermentation culture.

94. The method of any one of claims 87-93, comprising adding a salt solution to the whey protein to precipitate the whey protein to form the milk product.

95. The method of claim 94, wherein the salt solution comprises sodium chloride is at a concentration of between 50 micromolar and 2.5 M.

96. A method, comprising:
obtaining a milk product from a milk-producing mammal;
extracting a whey protein from the milk product;
forming cell culture media comprising the whey protein;
exposing a plurality of cells to the cell culture media; and
growing the plurality of cells in the cell culture media until the cells increase in size by an average of least 450%, relative to the size of the cells prior to exposing the cells to the cell culture media.

97. The method of claim 96, wherein the milk-producing mammal is a pregnant mammal.

98. The method of claim 96, wherein the milk-producing mammal is a nursing mammal.

99. The method of any one of claims 96-98, wherein the milk-producing mammal is artificially induced to produce the milk product.

100. The method of any one of claims 96-99, wherein the mammal is a cow.

101. The method of any one of claims 96-100, wherein the mammal is a goat.

102. The method of any one of claims 96-101, wherein the mammal is a sheep.

103. The method of any one of claims 96-102, comprising obtaining the milk product from the milk-producing mammal a plurality of times.

104. A method, comprising:
obtaining a milk product from a milk-producing organoid, wherein the organoid comprises a plurality of cultivated mammary epithelial cells;
extracting a whey protein from the milk product;
forming cell culture media comprising the whey protein; and
exposing a plurality of cells to the cell culture media.

105. A method, comprising:
obtaining a whey protein from a fermenting bacterial culture;
forming a cell culture media comprising the whey protein; and
exposing a plurality of cells to the cell culture media.

106. The method of claim 105, wherein at least some of the bacteria in the bacterial culture comprises at least one expression plasmid comprising a non-native milk-producing protein.

107. The method of claim 46, wherein the bacterial culture comprises E. coli.

108. The method of any one of claim 46 or 47, wherein the non-native milk-producing protein is a whey acidic protein.
